(19) United States
(12) Patent Application Publication
(10) Pub. No.: US 2025/0095858 A1
(43) Pub. Date: Mar. 20, 2025

(54) SYSTEMS AND METHODS FOR DETERMINING PERSISTENT DECIDUOUS TEETH RISK

(71) Applicant: Mars, Incorporated, McLean, VA (US)

(72) Inventors: Corryn Victoria WALLIS, Leicestershire (GB); Lucy Jane HOLCOMBE, Leicestershire (GB)

(21) Appl. No.: 18/467,939

(22) Filed: Sep. 15, 2023

(51) Int. Cl. G16H 50/30; G16H 50/70

(52) U.S. Cl. CPC G16H 50/30; G16H 50/70

(57) ABSTRACT

Various embodiments of this disclosure relate generally to predicting a risk level for the presence of persistent deciduous teeth (PDT) for one or more pets. The method comprises receiving, by one or more processors, pet data corresponding to a pet from a user device, the pet data including one or more pet attributes, based on the one or more pet attributes, determining, by the one or more processors, a result value indicating a PDT attribute weight for each of the one or more pet attributes, analyzing, by the one or more processors, the result value for each of the one or more pet attributes to determine a PDT risk level, the analyzing including utilizing a PDT risk level prediction algorithm, and displaying, by the one or more processors, the PDT risk level on one or more user interfaces of the user device.

### TECHNICAL FIELD

Various embodiments of the present disclosure relate generally to systems and methods for analyzing pet data to determine a risk of persistent deciduous teeth (PDT) for a pet.

### BACKGROUND

Persistent deciduous teeth (PDT), meaning those that fail to fall out at the appropriate time, are a common problem seen in pediatric canine dentistry. PDT cause issues in the oral cavity including malocclusion (misaligned bite), soft tissue trauma, and increased risk of a periodontal disease. Prompt identification and removal of the persistent deciduous tooth by a veterinarian, whilst avoiding damage to the underlying permanent tooth bud, may be recommended to avoid future damage. Additionally, the risk of PDT depends on a pet's attributes, such as the pet's size, breed, frequency of visits to a veterinarian, and the like. For example, small dogs may have a higher likelihood of PDT than big dogs. Early detection and mitigation of PDT may assist in preventing the increased risk of associated issues, such as a periodontal disease and malocclusion and help veterinarians and owners establish tailored oral care regimes for an individual pet. Therefore, a need exists for determining a PDT risk level for a pet.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, embodiments are disclosed for predicting a persistent deciduous teeth (PDT) risk level for one or more pets.

In one aspect, an exemplary embodiment of a method for predicting a persistent deciduous teeth (PDT) risk level for one or more pets is disclosed. The method may include receiving, by one or more processors, pet data corresponding to a pet from a user device, the pet data including one or more pet attributes. The method may include, based on the one or more pet attributes, determining, by the one or more processors, a result value indicating a PDT attribute weight for each of the one or more pet attributes. The method may include analyzing, by the one or more processors, the result value for each of the one or more pet attributes to determine a PDT risk level, the analyzing including utilizing a PDT risk level prediction algorithm. The method may include displaying, by the one or more processors, the PDT risk level on one or more user interfaces of the user device.

In one aspect, a computer system for predicting a persistent deciduous teeth (PDT) risk level for one or more pets is disclosed. The computer system may comprise at least one memory storing instructions and at least one processor configured to execute the instructions to perform operations. The operations may comprise receiving pet data corresponding to a pet from a user device, the pet data including one or more pet attributes. The operations may comprise, based on the one or more pet attributes, determining a result value indicating a PDT attribute weight for each of the one or more pet attributes. The operations may comprise analyzing the result value for each of the one or more pet attributes to determine a PDT risk level, the analyzing including utilizing a PDT risk level prediction algorithm. The operations may comprise displaying the PDT risk level on one or more user interfaces of the user device.

In one aspect, a non-transitory computer-readable medium storing instructions that, when executed by at least one processor, cause the at least one processor to perform operations for predicting a persistent deciduous teeth (PDT) risk level for one or more pets is disclosed. The operations may comprise receiving pet data corresponding to a pet from a user device, the pet data including one or more pet attributes. The operations may comprise, based on the one or more pet attributes, determining a result value indicating a PDT attribute weight for each of the one or more pet attributes. The operations may comprise analyzing the result value for each of the one or more pet attributes to determine a PDT risk level, the analyzing including utilizing a PDT risk level prediction algorithm. The operations may comprise displaying the PDT risk level on one or more user interfaces of the user device.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a block diagram of an exemplary system for analyzing data associated with a pet, according to one or more embodiments.

FIG. 2 depicts a flowchart illustrating an exemplary process for predicting a persistent deciduous teeth (PDT) risk level for one or more pets, according to one or more embodiments.

FIG. 3 illustrates an exemplary chart that categorizes canines both in breed size risk and individual risk of PDT, according to one or more embodiments.

FIG. 4 depicts an exemplary networked computing environment that may be utilized with techniques presented herein, according to one or more embodiments.

FIG. 5 depicts an example of a computing device that may execute the techniques described herein, according to one or more embodiments.

### DETAILED DESCRIPTION

According to certain aspects of the disclosure, methods and systems for analyzing pet data to determine a persistent deciduous teeth (PDT) risk for a pet are disclosed.

Dogs use their teeth for a multitude of tasks and, as such, it is vital for their health and wellbeing that their oral health is maintained. Persistent deciduous teeth (PDT), meaning those that fail to fall out at the appropriate time, can cause jaw misalignment, damage to the gums and an increased risk of gum disease. PDT cause issues in the oral cavity including malocclusion (misaligned bite), soft tissue trauma and increased risk of periodontal disease. Prompt identification and removal of the persistent deciduous tooth by a veterinarian, whilst avoiding damage to the underlying permanent tooth bud, may be recommended to avoid future damage. Additionally, the risk of PDT depends on a pet's attributes, such as the pet's size, breed, visits to a veterinarian, and the like. For example, small dogs may have a higher likelihood of PDT than big dogs. Additionally, for example, the odds of PDT significantly increase if it has been more than 2 years since the last professional dental scale and polish. The odds of PDT increase by about 50% when a dental scale and polish has been recorded 1-2 years previously versus less than one year.

Additionally, early detection and mitigation of PDT may assist in reducing the severity or delaying some of the consequences of PDT, such as malocclusion (misaligned bite), soft tissue trauma, increased risk of periodontal disease. Prompt identification can also assist in the early removal of the persistent deciduous tooth by a veterinarian, whilst avoiding damage to the underlying permanent tooth bud. Therefore, a need exists for determining a PDT risk level for a pet.

Therefore, the embodiments of the present disclosure are directed to systems and methods for predicting a persistent deciduous teeth (PDT) risk level for one or more pets. The systems and methods may include receiving, by one or more processors, pet data corresponding to a pet from a user device, the pet data including one or more pet attributes. The systems and methods may further include, based on the one or more pet attributes, determining, by the one or more processors, a result value indicating a PDT attribute weight for each of the one or more pet attributes. The systems and methods may further include analyzing, by the one or more processors, the result value for each of the one or more pet attributes to determine a PDT risk level, the analyzing including utilizing a PDT risk level prediction algorithm. The systems and methods may further include displaying, by the one or more processors, the PDT risk level on one or more user interfaces of the user device.

Advantages of such systems and methods may include improving the accuracy for predicting the PDT risk level by analyzing the pet's specific attributes, and then providing a personalized PDT risk level. Additional advantages may include increasing efficiency by utilizing prediction algorithms and machine-learning models. Further advantages may include assisting pet owner's in the identification of PDT and corresponding ailments and providing personalized recommendations to the pet owner.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The terms “pet” and “household pet” as used in accordance with the present disclosure can refer to, without limitation, domesticated or tamed animals such as, e.g., dogs, cats, rabbits, horses, and the like.

The term “pet owner” may include, for example, without limitation, any person, organization, and/or collection of persons that owns and/or provides food and shelter for a pet. For example, a “pet owner” may include a pet adopter, a pet caretaker, a pet caregiver, and an animal shelter.

The term “veterinarian” may include, for example, without limitation, any person, organization, and/or collection of persons that provides medical care to a pet. For example, a “veterinarian” may include a veterinary technician, a veterinary personnel, and a veterinarian practitioner.

The terms “canine” and “dog” may include, for example, without limitation, recognized dog breeds (some of which may be further subdivided). For example, the recognized dog breeds may include Afghan hound, Airedale, akita, Alaskan malamute, basset hound, beagle, Belgian shepherd, bloodhound, border collie, border terrier, borzoi, boxer, bulldog, bull terrier, cairn terrier, Chihuahua, chow, cocker spaniel, collie, corgi, dachshund, Dalmatian, Doberman, English setter, fox terrier, German shepherd, golden retriever, great dane, greyhound, griffon bruxellois, Irish setter, Irish wolfhound, King Charles spaniel, Labrador retriever, Lhasa apso, mastiff, Newfoundland, old English sheepdog, Papillion, Pekingese, pointer, Pomeranian, poodle, pug, Rottweiler, St. Bernard, saluki, Samoyed, schnauzer, Scottish terrier, Shetland sheepdog, shih Tzu, Siberian husky, Skye terrier, springer spaniel, West Highland terrier, Yorkshire terrier, etc.

As used herein, the terms “pet data” or “pet metadata” may include, for example, without limitation, biological data, such as, any one or combination of certain biological information or attributes of a pet including at least its breed, age, size, weight, body condition, shape of head (e.g., skull shape), predicted size category, genetic markers that are associated with a higher risk of PDT, predicted weight as an adult, and/or oral health data related to common symptoms of PDT. Pet data may also include environmental data (e.g., external factors). Environmental data may include information regarding the frequency a pet visits the vet, the pet's last oral assessment, or the pet's oral care routine, such as the frequency of tooth brushing and/or the use of dental treats, oral rinses, oral gels, and chew toys. Other environmental data may include changes in pet behavior (e.g., eating habits and pawing at the face), which may indicate PDT. Additionally, for example, the pet data may include answers to questions pertaining to, but not limited to, the biological information, environmental/external factors, and attributes discussed above.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. Any suitable type of training may be used, e.g., stochastic, gradient boosted, random seeded, recursive, epoch or batch-based, etc.

The term “diagnosis” may include, for example, without limitation, the recognition and/or identification of a disease or condition (e.g., PDT), the prediction of the course of a disease or condition, the prediction of the likelihood of the disease or condition, as well as a conclusion with respect to a risk level associated with a disease or condition (e.g., low risk, medium risk, and high risk).

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

### Exemplary Embodiment for Analyzing Data Associated with a Pet

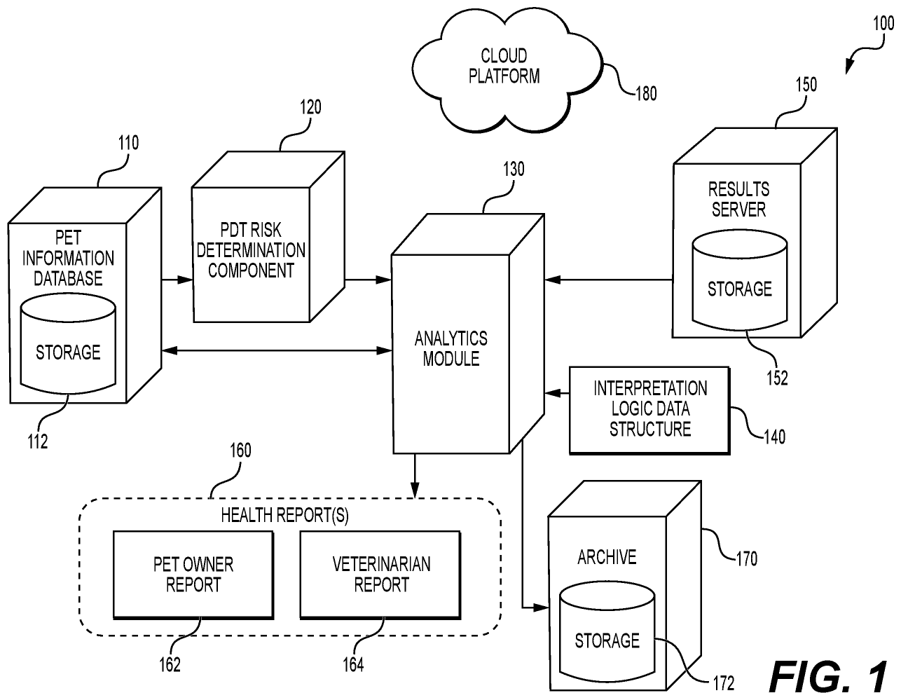

FIG. 1 shows an exemplary embodiment of a system 100 for analyzing data associated with a pet. In general, system 100 may comprise pet information database 110, PDT risk determination component 120, analytics module 130, interpretation logic data structure 140, results server 150, health report(s) 160, archive 170, and cloud platform 180.

System 100 may be implemented on a cloud platform 180, allowing for the transmission or sharing of data between each of pet information database 110, PDT risk determination component 120, analytics module 130, interpretation logic data structure 140, results server 150, health report(s) 160, and archive 170 in a cloud environment.

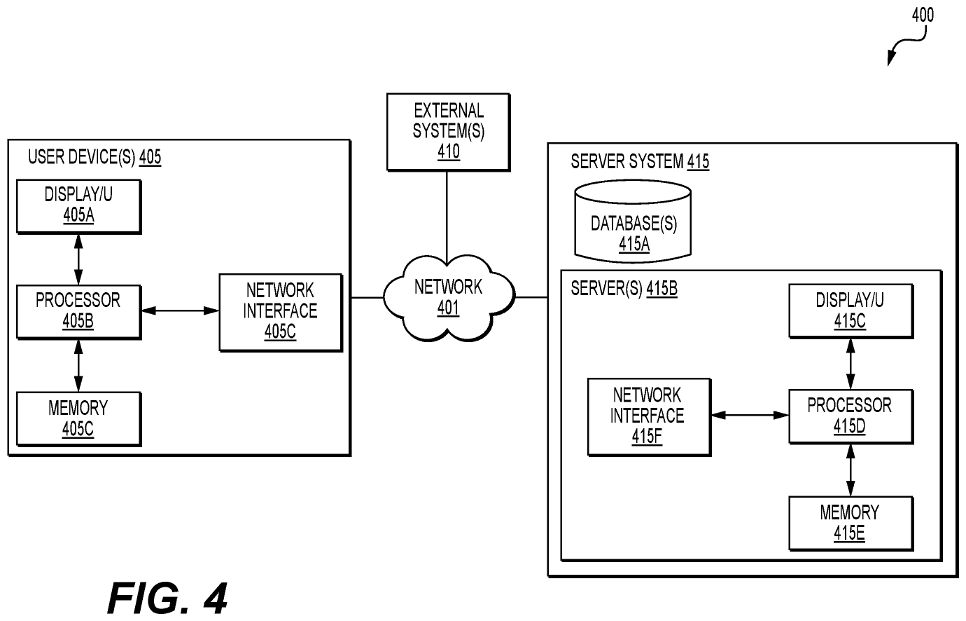

Pet information database 110 comprises storage 112. Storage 112 in pet information database 110 may contain pet data (e.g., biological and environmental data) associated with a pet. One or more users may access pet information database 110 by a server via one or more user devices. The one or more users may include a pet owner and a veterinarian. The one or more users may use one or more user devices to input pet data associated with a pet. A user device may be a device consistent with the computing device depicted in FIG. 4, such as a desktop computer, a tablet, a laptop, a smart phone, a smart watch, etc.

The pet data associated with a pet and input by one or more users into pet information database 110 may include one or more of the following attributes of a pet: a pet type (e.g., breed, pet category), a pet head shape, a pet weight size category, a pet weight, a pet body condition, a predicted pet size category, a pet adult weight category, a pet age, a pet medical (e.g., veterinarian) interaction frequency, a pet plan, or a last medical (e.g., veterinarian) interaction. Additional pet data, which may be input by the one or more users and stored in pet information database 110, may include oral health data pertaining to common symptoms and consequences of PDT and information pertaining to a pet's oral care routine. For example, the one or more users may input information regarding whether the pet has malocclusion (misaligned bite), trapped food debris, soft tissue trauma, swollen, inflamed, reddened, and/or bleeding gums, a change in behavior (e.g., change in eating habits or pawing at the face), a sensitive stomach, halitosis, plaque, or tartar build-up, as well as the frequency of tooth brushing and use of dental treats, oral rinses, and/or oral gels. The pet data associated with a pet may be stored in storage 112 of pet information database 110.

In some embodiments, both a pet owner and a veterinarian may input pet data associated with a pet in pet information database 110. In other embodiments, only one of a pet owner and a veterinarian may input pet data associated with a pet in pet information database 110. In at least some embodiments, the one or more users may be prompted to input at least one of the following attributes of a pet in pet information database 110: a pet type (e.g., breed, pet category), a pet head shape, a pet weight size category, a pet weight condition, a pet body condition, a predicted pet size category, a pet adult weight category, a pet age, a pet medical (e.g., veterinarian) interaction frequency, a pet plan, a last medical (e.g., veterinarian) interaction, as well as any other pet data described in this disclosure (e.g., the data described in the previous paragraph). In some examples, the one or more users may be prompted to input a halitosis status of absent or present in pet information database 110.

Pet information database 110 may transmit pet data associated with a pet to PDT risk determination component 120. Pet information database 110 may also transmit pet data associated with a pet to analytics module 130. In some embodiments, pet information database 110 may also receive data, such as one or more results statements related to a pet's oral health, from analytics module 130.

PDT risk determination component 120 may receive pet data associated with a pet from pet information database 110, as discussed above. PDT risk determination component 120 may be deployed in an application programming interface (API). PDT risk determination component 120 may also transmit PDT risk data to analytics module 130. PDT risk determination component 120 as described herein may also be used on other applications (e.g., websites) outside of system 100.

Results server 150 comprises storage 152. A user may input results, which are obtained from one or more tests or samples collected from a pet, onto results server 150 via user device. For example, the results may relate to determining the breed of a pet. The results may relate to new biomarkers. For example, the biomarkers may include genetic markers that may identify the pet's genetic predisposition to having PDT. The results may be stored in storage 152. Results server 150 may transmit the results to analytics module 130.

Analytics module 130 may receive pet data associated with a pet from one or more data sources in system 100 and results data associated with the pet from results server 150. For example, analytics module 130 may receive pet data associated with a pet directly from pet information database 110 as well as from PDT risk determination component 120. In some embodiments, select pet data associated with a pet may be transmitted from pet information database 110 to PDT risk determination component 120 for analysis, and the PDT risk data that is generated based on the analysis may be transmitted to analytics module 130. In some embodiments, certain types of pet data, such as, e.g., pet veterinarian interaction frequency, may be transmitted directly from pet information database 110 to analytics module 130, without first being transmitted to PDT risk determination component 120.

Analytics module 130 may use interpretation logic data structure 140 to analyze the pet data received with the results received. Analytics module 130 may determine one or more statements related to a pet's oral health based on the analysis of the pet's pet data with the pet's results using interpretation logic data structure 140, and may generate health report(s) 160 containing the one or more statements. Health report(s) 160 may include pet owner report 162 and/or veterinarian report 164. Analytics module 130 may also transmit/store the one or more statements generated to archive 170. Archive 170 may store the analyzed data received from analytics module 130 in storage 172.

It should also be noted that, although system 100 depicts the use of interpretation logic data structure 140 by analytics module 130, in some embodiments, analytics module 130 may use a machine-learning model. In these embodiments, a trained machine learning model may be stored and used by analytics module 130.

### Exemplary Method for Predicting a PDT Risk Level

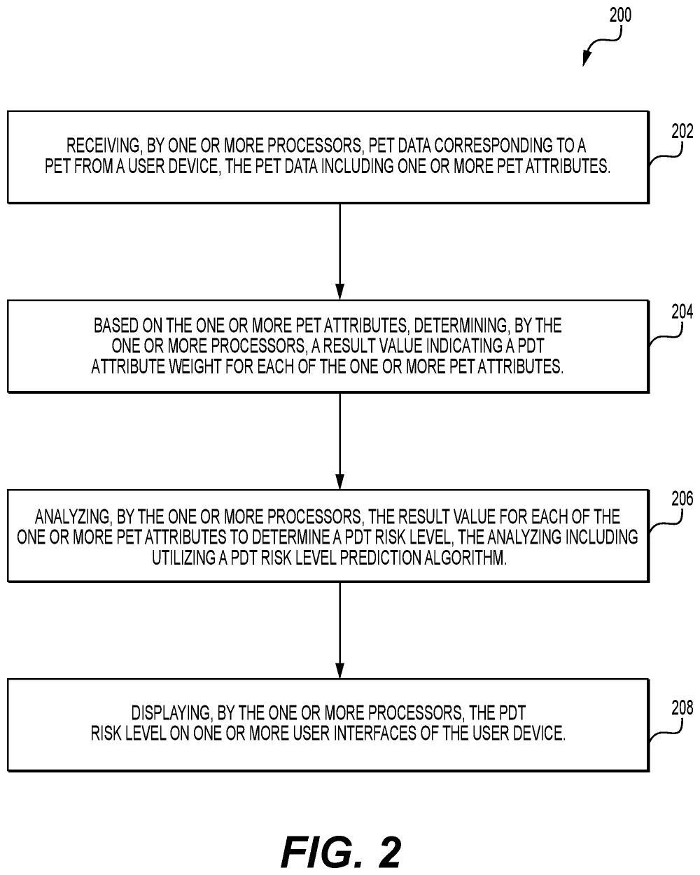

FIG. 2 depicts a flowchart illustrating an exemplary method 200 for predicting a persistent deciduous teeth (PDT) risk level for one or more pets, according to one or more embodiments. In some embodiments, PDT risk determination component 120 may perform the exemplary method 200. In addition, method 200 may be performed by one or more processors of a server that is in communication with one or more user devices and other external system(s) via a network. However, it should be noted that method 200 may be performed by any one or more of the servers, one or more user devices, or other external systems.

The method may include receiving, by one or more processors, pet data corresponding to a pet from a user device, the pet data including one or more pet attributes (Step 202). In some embodiments, PDT risk determination component 120 may receive the pet data from a user device and/or one or more databases (e.g., pet information database 110). The pet data may include at least one of: an image of the pet, a medical record corresponding to the pet, pet data input via the user device, or pet data stored in one or more pet data stores. The image of the pet may include a digital photograph or dental radiograph of the pet's teeth, face, and/or body. The medical record may include information regarding the pet's medical history, such as oral examinations, vaccinations, medications, hospitalizations, diagnoses, and the like. A pet owner, veterinarian, medical professional, and/or third-party may input the pet data. Additionally, or alternatively, the pet data may have been previously stored in one or more data stores (e.g., pet information database 110).

The pet data may include one or more pet attributes (and corresponding pet attribute values) that may include one or more of: a pet type, a pet head shape, a pet weight size category, a pet weight condition, a pet body condition, a predicted pet size category, a pet adult weight category, a pet age, a pet medical (e.g., veterinarian) interaction frequency, a pet plan, a last medical (e.g., veterinarian) interaction, behavior motion data, historical pet data, pet household information, as well as any other information regarding any aspect of the pet's life, health (e.g., diet and oral care regimes), or circumstances. The pet type may correspond to one or more breeds. Additionally, or alternatively, the pet type may correspond to a pet category, such as dog, cat, bird, rabbit, horse, and the like. The pet head shape may correspond to the shape of the pet's head. The pet weight size category may include at least one of: an extra-small category, a small category, a medium-small category, a medium-large category, a large category, or an extra-large category. The pet weight may include at least one of: underweight, average weight, overweight, obese, and the like. Additionally, or alternatively, the pet weight may include a numerical number corresponding to the weight of the pet. The pet body condition may include a pet body condition score (“BCS”). For example, the BCS may include a numerical value (e.g., 0-9 on a 9 point scale). The BCS may be recorded on a 5-point scale (e.g., 1-malnourished, 3=normal, 5=obese) or entered as a diagnosis of overweight/obese or underweight/emaciation. Additionally, for example, if there are multiple conflicting BCS for the same day, the non-normal BCS may be used (e.g., a pet's visit had a BCS entry of “normal” and diagnosis of overweight, this pet may be considered overweight on that visit). Additionally, or alternatively, the BCS may be converted into a three-point scale (e.g., 1=underweight, 2=ideal, 3=overweight).

The predicted pet size category may correspond to the predicted size of the pet when the pet is full-grown. The pet adult weight category may correspond to the weight of the pet when the pet is full-grown. The pet age may correspond to the age of the pet. The pet medical (e.g., veterinarian) interaction frequency may correspond to how frequently the pet visits the veterinarian. The pet plan may correspond to the pet's wellness plan. The last medical (e.g., veterinarian) interaction may correspond to the last time the pet visited the veterinarian, such as the medical data of the pet visit. The behavior motion data may describe how the pet walks, sits, runs, eats, and the like. The historical pet data may include previously input pet data, as well as data that describes the pet in the past. The pet household information may describe where the pet lives, other pets that live with the pet, other people that live with the pet, and the like. In some embodiments, the one or more pet attributes may include result data (e.g., results stored in results server 150).

In some embodiments, PDT risk determination component 120 may receive the one or pet more attributes from one or more user devices. For example, PDT risk determination component 120 may prompt one or more users (e.g., a pet owner, a veterinarian) to input the one or more pet attributes via a user interface rendered on one or more user devices. Some of the pet attributes, such as age and weight, may be provided by a pet owner via the user device, while other pet attributes, such as predicted size category, shape of the head, and/or the predicted weight as an adult, may be provided by a veterinarian via the user device.

The method may include, based on the one or more pet attributes, determining, by the one or more processors, a result value indicating a PDT attribute weight for each of the one or more pet attributes (Step 204). Each of the pet attributes may be analyzed individually or together to determine a PDT attribute weight for the attribute, where the PDT attribute weight may be expressed as a result value (e.g., a percentage, a ratio, a number, and the like). Each attribute may have a corresponding PDT probability weight, where the PDT probability weight may indicate the likelihood that the pet may be diagnosed with PDT. For example, a pet attribute may include a pet age of 1 year old, where a corresponding result value may include a PDT attribute weight of 11%. In some embodiments, one or more databases may store result values corresponding to the pet attributes, where the system may retrieve the result value from the one or more databases.

In some embodiments, a machine-learning model may analyze the one or more pet attributes to determine the result value indicating the PDT attribute weight. The one or more pet attributes may be input into the machine-learning model, where the machine-learning model may output the result value for such attribute(s). Additionally, the machine-learning model may have been previously trained to determine the PDT attribute weight for one or more pet attributes.

The method may include analyzing, by the one or more processors, the result value for each of the one or more pet attributes to determine a PDT risk level, the analyzing including utilizing a PDT risk level prediction algorithm (Step 206). The PDT risk level may be expressed as a percentage, a ratio, a number, a color, a phrase (e.g., “low risk,” “medium risk,” or “high risk”) and the like.

In some embodiments, analyzing the result value to determine the PDT risk level may include receiving, by the one or more processors, one or more pet dental datasets for one or more similar pets from one or more data stores, wherein the one or more similar pets have at least one similar attribute to the pet. The data stores may store datasets for a plurality of other pets, where the datasets may include attributes (including corresponding attribute values) for each of the other pets. The attributes may include one or more of: a pet type (e.g., breed, pet category), a pet head shape, a pet weight size category, a pet weight condition, a pet body condition, a predicted pet size category, a pet adult weight category, a pet age, a pet medical (e.g., veterinarian) interaction frequency, a pet plan, or a last medical (e.g., veterinarian) interaction, as previously described. The datasets may also include additional pet information that describes the other pets, as well as a PDT risk level for such pet. The method may include receiving and analyzing the datasets to determine at least one dataset of a similar pet. The dataset may include at least one similar attribute to the initial pet. For example, the pet data may include an age attribute of 1 years old, and the similar pet dataset may include an age attribute of 1 years old and a PDT risk level of 0.25.

Additionally, analyzing the result value to determine the PDT risk level may further include utilizing, by the one or more processors, the PDT risk level prediction algorithm to determine the PDT risk level by comparing the result value corresponding to each of the one or more pet attributes with the one or more pet dental datasets. The PDT risk level prediction algorithm may predict the risk of PDT in pets based on the pet attributes. For example, the PDT risk level prediction algorithm may analyze the dental datasets to determine which datasets include attributes that are the most similar to the attributes of the pet. The PDT risk level prediction algorithm may also analyze the corresponding PDT risk level of such datasets to determine the PDT risk level for the pet. The PDT risk level may include a low risk, a medium risk, or a high risk, where the PDT risk level may indicate a probability of the pet being diagnosed with PDT. Additionally, or alternatively, the PDT risk level may include a score indicating the PDT attribute weight.

For example, where the percentage total of the pet datasets whose breed indicating PDT is in excess of 8%, a score of 3 may be allocated in the PDT risk level prediction algorithm applied to the initial pet. A score of 2 may be allocated in the PDT risk level prediction algorithm for the pet datasets whose breed shows between 2-5% PDT, and a score of 1 may be allocated in the PDT risk level prediction algorithm for the pet datasets whose breed shows PDT in less than 2% of the total population. In some embodiments, the score may be represented as high risk, medium risk, and low risk pets.

Utilizing the PDT risk level prediction algorithm may include utilizing a machine-learning model, where the machine-learning model may have been trained to determine the PDT risk level. For example, the machine-learning model may have been trained using one or more training result values of one or more attributes, one or more training datasets, and/or one or more training PDT risk levels to learn associations between the result values, datasets, and/or PDT risk levels. The machine-learning model may receive the result values for each of the one or more pet attributes, as well as the one or more pet dental datasets. The machine-learning model may analyze and compare the result values and the dental datasets to determine a PDT risk level.

The method may include displaying, by the one or more processors, the PDT risk level on one or more user interfaces of the user device (Step 208). The one or more user interfaces may display the PDT risk level as “low risk,” “medium risk,” or “high risk.” Additionally, or alternatively, the one or more user interfaces may display a numerical value. For example, the one or more user interfaces may display “0.1—Low Risk.” The one or more user interfaces may display the PDT risk level utilizing different colors or graphics, which may depend on the PDT risk level. For example, the one or more user interfaces may display “Low Risk” in a green color, “Medium Risk” in a yellow color, or “High Risk” in a red color. In some embodiments, the displaying may include displaying a notification on the one or more user interfaces of the user device. The notification may alert the user that the pet requires medical attention and/or medical treatment.

In some embodiments, the PDT risk level may be stored in one or more databases. The PDT risk level may be stored with a corresponding unique pet identifier. In future analysis, the PDT risk level prediction algorithm may utilize the stored PDT risk level when performing the PDT risk level analysis. Additionally, or alternatively, one or more stored PDT risk levels may be analyzed in combination with a current PDT risk level to determine whether the pet's PDT risk level is increasing or decreasing. The one or more user interfaces may display a notification indicating whether the pet's PDT risk level has increased or decreased.

In some embodiments, the method may further include determining, by the one or more processors, one or more recommendations based on the PDT risk level. For example, if the PDT risk level is a “high risk,” the recommendation may include recommendations for how to manage or treat PDT, such as recommending that the pet receives regular dental examinations or recommending that the vet remove or monitor a tooth. Additionally, for example, if the PDT risk level is a “medium risk,” the recommendation may include more frequent scale and polish of the pet's teeth to help in preventing or delaying periodontal disease. The recommendations may include links to internal or external sources that may provide support for how to identify and treat PDT. The method may further include displaying, by the one or more processors, the one or more recommendations on the one or more user interfaces of the user device. For example, the one or more user interfaces may display the links to the internal or external sources. Additionally, or alternatively, the one or more user interfaces may display the recommendations.

Although FIG. 2 shows example blocks of exemplary method 200, in some implementations, the exemplary method 200 may include additional blocks, fewer blocks, different blocks, or differently arranged blocks than those depicted in FIG. 2. Additionally, or alternatively, two or more of the blocks of the exemplary method 200 may be performed in parallel.

### Exemplary PDT Risk Score Chart

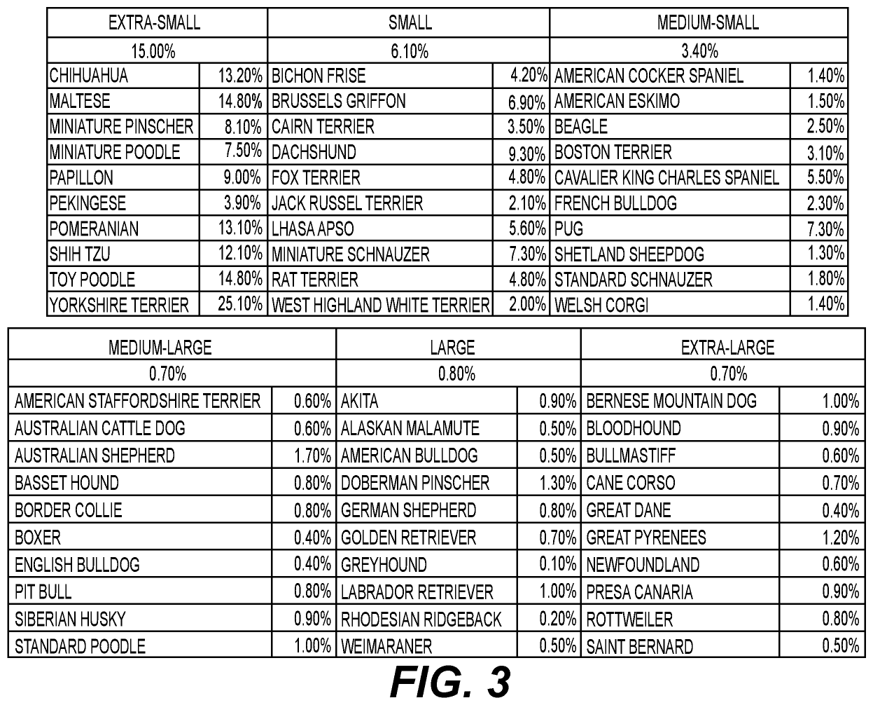

FIG. 3 illustrates an exemplary chart that categorizes canines both in breed size risk and individual breed risk of PDT. Specifically, the chart illustrates the risk of PDT for each individual breed, as well as for the breed size. The size groups include the following: extra-small, small, medium-small, medium-large, large, and extra-large. The chart illustrates how the PDT risk score decreases for each canine size group, as the canine's size increases. A risk score higher than 10% may indicate a high risk of PDT. For example, a toy poodle that has a high risk of PDT may have a PDT risk score of 14.8%. A risk score between 2-10% may indicate a medium risk of PDT. For example, a dachshund that has a medium risk of PDT may have a PDT risk score of 9.3%. A risk score less than 2% may indicate a low risk of PDT. For example, a boxer that has a low risk of PDT may have a PDT risk score of 0.4%.

### Exemplary Data

Medical records collected from almost 3 million dogs (representing 60 breeds) visiting a chain of veterinary hospitals across the United States over a 5-year period showed an overall prevalence of 7% for PDT, with extra-small breeds (<6.5 kg) showing significantly higher prevalence (15%) than all other breed sizes (P<0.001). Statistical modelling of extra-small, small and medium-small breeds showed that those on Wellness Plans (“WP”) or that had not received a dental prophylaxis for at least two years showed significantly increased odds of PDT (P<0.0001). Under-conditioned dogs in the extra-small, small or medium-small size categories had a slightly decreased odds of PDT (OR 0.57-0.89., P<0.0001) whereas those that were over-conditioned had a slightly increased odds (OR 1.11-1.60, P<0.0001).

### Statistical Analysis

Specifically, data were analysed using software. Descriptive statistics for all breed size categories as well as for individual breeds were calculated. The prevalence of PDT by size category and breed was calculated based on whether a pet had at least one PDT during the 5-year study period. Model for the outcome “to have at least one persistent deciduous tooth before age of 60 months” was performed via logistic regression.

Logistic regression was used to model PDT by breed size category. Only the extra-small, small and medium-small size categories were used as these had the highest proportions of PDT. For the breed size category model, extra-small was used as the reference with covariates and their interactions. Covariates included dental cleaning and average pet weight during 5-year study period, dental cleaning and on WP at time of visit and time on WP in months. Dummy variables small and medium-small, body condition score, sex, neuter status and days since last dental cleaning were also included. Odds ratios (OR; with confidence limits) for the individual terms were calculated.

Pairwise comparisons with Bonferroni correction were undertaken to compare estimated OR between extra small, small and medium-small size categories, as well as individual breeds within their size category. For the size category analysis, extra-small was used as the reference with the following covariates (without interactions): dental scale and polish during study period, dental scale and polish at visit, pet age (months), normalised pet weight (pet weight-median/median), Wellness Plan (WP) at time of visit, duration of WP (months), dummy variables for BCS: over-conditioned, under-conditioned (normal as reference), dummy for gender: female (male as reference), dummy for spay/neutered status: neutered (intact as reference), dummies for time since last dental scale and polish: less than 2 years, more than 2 years, none recorded (<1 year as reference). For the breeds within each size category analysis, Yorkshire terrier was used as the reference for the extra-small category, Dachshund for the small category and Pug for the medium-small category. Results were considered to be significant if P<0.017 for the breed size category comparisons and P<0.001 for the breeds within their size category based on Bonferroni correction for multiplicity.

A final analysis of presence of PDT over the entire five-year time scale was performed for size categories extra-small, small and medium-small using a generalized estimating equation (GEE) model with compound symmetry working covariance structure to correct for repeated measures per pet.

### Results

### Study Population

A total of 5,787,581 dogs were seen across 31,306,476 visits. Of these, 3,320,519 dogs (57.4%) and 18,233,668 visits (58.4%) were from 60 pure breeds. After applying exclusion criteria, 2,841,032 dogs and 14,746,685 visits entered the analysis.

Most dogs (36.9%) were in the extra-small breed weight category. The other breed weight categories comprised between 11.7% and 18.4% of dogs except for the extra-large category which consisted of the remaining 3.1% of dogs.

The average age of the study population overall was 61.8 months (+44.1 months), with an average age at first visit of 50.9 months (+44.0). There were slightly more male dogs (52.6%) than female dogs (47.4%) and the majority (70.8%) were neutered/spayed. Overall, the average weight of each size category was: 4.7 kg (extra-small), 7.6 kg (small), 12.1 kg (medium-small), 26.1 kg (medium-large), 34.3 kg (large) and 45.9 kg (large). In total, 83.1% of dogs were deemed to have an ideal BCS at the start of the study. A small percentage of dogs were recorded as under-conditioned (mean total 2.4%). The extra-large breed weight category had the largest percentage of dogs (4.2%) with a BCS recorded as under-conditioned at first visit whereas the small and medium-small breed size categories had the lowest (both 1.4%). There was also a high proportion of dogs recorded as over-conditioned (14.5%). The small (17.9%), medium-small (22.9%) and large (18.7%) breed size categories had the greatest percentage of dogs that were recorded as over-conditioned at first visit.

Over half the dogs (54.6-58.6%) had been enrolled on a WP within the five-year study period and the average time spent on the WP was 17.4 months. Around 81.2% of the in-patient visits at the veterinary hospital were in connection with a WP.

On average, 30.8% of dogs had received a dental scale and polish procedure in the last 12 months prior to the visit, with the highest percentage in the small dog category (39.0%) and lowest in extra-large (21.8%). Overall, an average of 2.2% of dogs had not received a dental scale and polish for over 24 months, with the small size category showing the highest average of 2.8%.

### Prevalence of Persistent Deciduous Teeth

The overall mean prevalence of PDT for the 60 breeds of dog studied over the five-year study period was 7.0%, with highest prevalence in the extra-small dog category (15.0%), followed by small (6.1%) and medium-small (3.4%), as shown in FIG. 3. Medium-large, large and extra-large breed categories each had a mean prevalence of <1% PDT.

In terms of the individual breeds, Yorkshire terriers had the highest prevalence of PDT (25.1%), followed by Maltese and toy poodle (both 14.8%). The top 10 breeds in terms of persistent deciduous tooth prevalence comprised nine extra-small breeds and one small breed (Dachshund). Two other breeds showing a persistent deciduous tooth prevalence rate of more than the overall mean of 7% were the miniature schnauzer (7.3%) and pug (7.3%). The Pekingese was the only extra-small breed that was below the overall average prevalence, at 3.8%. A prevalence of ≤1.0% PDT was reported in 27 breeds, all medium-large, large or extra-large. The lowest prevalence recorded was 0.1% in the greyhound. PDT prevalence figures for all 60 breeds over the five-year study period are shown in FIG. 3.

Regression analysis modelling using GEE across the entire five-year period showed all covariates, with the exception of female, had a small (OR=0.98-1.11) but statistically significant effect on the PDT outcome (all P<0.0001). The statistical significance, however, was likely a result of the extensive data and therefore unlikely to be of clinical relevance. Comparison of breed size categories across the entire five-year period showed that small and medium-small breeds had a lower probability of PDT versus extra-small breeds (OR=0.36 and 0.20 respectively, P<0.001).

Pairwise comparisons between extra-small, small and medium-small breed categories showed that the small and medium-small categories had significantly lower odds of PDT versus extra-small (OR=0.41, 0.20 respectively, P<0.0001). Medium-small dogs also had lower odds of PDT when compared with small (OR=0.48; P<0.0001).

From the statistical model of breeds within the extra-small category, pairwise comparisons showed that the Yorkshire terrier had significantly higher odds of PDT versus all other extra-small breeds (OR=1.9-7.2, P<0.0001). The Pekingese had significantly lower odds of PDT versus all other breeds in the category (OR=0.14-0.56, P<0.0001).

In the small size category, Dachshunds had a significantly higher odds of PDT compared to all other breeds within the size category (OR=1.21-4.21, P<0.0001). Likewise, miniature schnauzer had an increased odds of PDT versus all other breeds within the category with the exception of Dachshund (OR=1.1-3.48, P<0.0001). West Highland white terrier had a significantly lower odds of PDT versus all other breeds within the size category (OR=0.24-0.85, P<0.0001).

In the medium-small size category, Pug had an increased PDT versus all other breeds within the category (OR=1.62-6.73, P<0.0001). Cavalier King Charles spaniel also had higher odds of PDT compared to all other breeds within the size category with the exception of pug (OR=1.56-4.15, P<0.0001). Standard schnauzer had the lowest odds of PDT versus all other breeds within the medium-small category although not significant versus all other breeds within the size category after Bonferroni correction (OR=0.15-0.76, P=0.001). This finding may have been due to the under representation of standard schnauzers which meant there may not have been large enough numbers of individuals to detect a significant difference.

### Risk Factors for Persistent Deciduous Teeth

According to the breed size category model, the odds of PDT were significantly increased if it was more than 2 years since the last professional dental scale and polish (OR=3.36, 2.70 and 2.17 for the extra-small, small and small-medium size categories respectively, all P<0.0001). The odds of PDT were increased by around 50% when a dental scale and polish had been recorded 1-2 years previously versus <1 year (OR=1.51-1.62, P<0.0001). When the last dental scale and polish procedure was recorded as unknown, this had very little influence on the odds of PDT (0.99-1.16; P<0.0001).

The breed size category model showed a significant effect of weight on the probability of PDT. Dogs with a body weight further from the median weight for their breed size category had a decreased odds of PDT (OR-0.21, 0.16 and 0.14 respectively, all P<0.0001). For each individual breed within the extra-small, small and medium-small size categories, all showed OR<0.29 (P<0.0001) in association with their body weight being further from the median weight for the breed. The size category model indicated that being under-conditioned slightly decreased the odds of PDT tooth diagnosis in extra-small, small or medium-small dogs (OR=0.85, 0.89 and 0.57 respectively, P<0.0001). In contrast, being over-conditioned slightly increased the odds of PDT in extra-small, small and medium-small dogs (OR=1.11, 1.45 and 1.60 respectively, P<0.0001).

### Exemplary Environment and Exemplary Device

FIG. 4 depicts an exemplary environment 400 that may be utilized with techniques presented herein. One or more user device(s) 405, one or more external system(s) 410, and one or more server system(s) 415 may communicate across a network 401. As will be discussed in further detail below, one or more server system(s) 415 may communicate with one or more of the other components of the environment 400 across network 401. The one or more user device(s) 405 may be associated with a user.

In some embodiments, the components of the environment 400 are associated with a common entity, e.g., a veterinarian, a mobile platform, or the like. In some embodiments, one or more of the components of the environment is associated with a different entity than another. The systems and devices of the environment 400 may communicate in any arrangement. As will be discussed herein, systems and/or devices of the environment 400 may communicate in order to one or more of: generate, train, and/or use a machine-learning model to predict a PDT risk level, among other activities.

The user device 405 may be configured to enable the user to access and/or interact with other systems in the environment 400. For example, the user device 405 may be a computer system such as, for example, a desktop computer, a mobile device, a tablet, etc. In some embodiments, the user device 405 may include one or more electronic application(s), e.g., a program, plugin, browser extension, etc., installed on a memory of the user device 405.

The user device 405 may include a display/user interface (UI) 405A, a processor 405B, a memory 405C, and/or a network interface 405D. The user device 405 may execute, by the processor 405B, an operating system (O/S) and at least one electronic application (each stored in memory 405C). The electronic application may be a desktop program, a browser program, a web client, or a mobile application program (which may also be a browser program in a mobile O/S), an applicant specific program, system control software, system monitoring software, software development tools, or the like. For example, environment 400 may extend information on a web client that may be accessed through a web browser. In some embodiments, the electronic application(s) may be associated with one or more of the other components in the environment 400. The application may manage the memory 405C, such as a database, to transmit streaming data to network 401. The display/UI 405A may be a touch screen or a display with other input systems (e.g., mouse, keyboard, etc.) so that the user(s) may interact with the application and/or the O/S. The network interface 405D may be a TCP/IP network interface for, e.g., Ethernet or wireless communications with the network 401. The processor 405B, while executing the application, may generate data and/or receive user inputs from the display/UI 405A and/or receive/transmit messages to the server system 415, and may further perform one or more operations prior to providing an output to the network 401.

External systems 410 may be, for example, one or more third party and/or auxiliary systems that integrate and/or communicate with the server system 415 in performing various PDT risk level prediction tasks. External systems 410 may be in communication with other device(s) or system(s) in the environment 400 over the one or more networks 401. For example, external systems 410 may communicate with the server system 415 via API (application programming interface) access over the one or more networks 401, and also communicate with the user device(s) 405 via web browser access over the one or more networks 401.

In various embodiments, the network 401 may be a wide area network (“WAN”), a local area network (“LAN”), a personal area network (“PAN”), or the like. In some embodiments, network 401 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 415 may include an electronic data system, e.g., a computer-readable memory such as a hard drive, flash drive, disk, etc. In some embodiments, the server system 415 includes and/or interacts with an application programming interface for exchanging data to other systems, e.g., one or more of the other components of the environment.

The server system 415 may include a database 415A and at least one server 415B. The server system 415 may be a computer, system of computers (e.g., rack server(s)), and/or or a cloud service computer system. The server system may store or have access to database 415A (e.g., hosted on a third party server or in memory 415E). The server(s) may include a display/UI 415C, a processor 415D, a memory 415E, and/or a network interface 415F. The display/UI 415C may be a touch screen or a display with other input systems (e.g., mouse, keyboard, etc.) for an operator of the server 415B to control the functions of the server 415B. The server system 415 may execute, by the processor 415D, an operating system (O/S) and at least one instance of a servlet program (each stored in memory 415E).

The server system 415 may generate, store, train, or use a machine-learning model, configured to predict a PDT risk level for a pet. The server system 415 may include a machine-learning model and/or instructions associated with the machine-learning model, e.g., instructions for generating a machine-learning model, training the machine-learning model, using the machine-learning model, etc. The server system 415 may include instructions for predicting a PDT risk level for a pet, e.g., based on the output of the machine-learning model, and/or operating the display 415C to output a PDT risk level, e.g., as adjusted based on the machine-learning model. The server system 415 may include training data, e.g., one or more training result values, one or more training datasets, and/or one or more training PDT risk levels.

In some embodiments, a system or device other than the server system 415 is used to generate and/or train the machine-learning model. For example, such a system may include instructions for generating the machine-learning model, the training data and ground truth, and/or instructions for training the machine-learning model. A resulting trained machine-learning model may then be provided to the server system 415.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between result values, datasets, and/or PDT risk levels.

In various embodiments, the variables of a machine-learning model may be interrelated in any suitable arrangement in order to generate the output. For example, the machine-learning model may include one or more convolutional neural network (“CNN”) configured to identify PDT risk levels, and may include further architecture, e.g., a connected layer, neural network, etc., configured to determine a relationship between the identified features in order to determine a predicted PDT risk level for a pet.

Although depicted as separate components in FIG. 4, it should be understood that a component or portion of a component in the environment 400 may, in some embodiments, be integrated with or incorporated into one or more other components. For example, a portion of the display 415C may be integrated into the user device 405 or the like. In some embodiments, operations or aspects of one or more of the components discussed above may be distributed amongst one or more other components. Any suitable arrangement and/or integration of the various systems and devices of the environment 400 may be used.

Further aspects of the machine-learning model and/or how it may be utilized to predict a PDT risk level are discussed in further detail in the methods above. In the methods, various acts may be described as performed or executed by a component from FIG. 4, such as the server system 415, the user device 405, or components thereof. However, it should be understood that in various embodiments, various components of the environment 400 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

In general, any process or operation discussed in this disclosure that is understood to be computer-implementable, such as the processes illustrated in FIG. 2, may be performed by one or more processors of a computer system, such any of the systems or devices in the environment 400 of FIG. 4, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 4. One or more processors of a computer system may be included in a single computing device or distributed among a plurality of computing devices. A memory of the computer system may include the respective memory of each computing device of the plurality of computing devices.

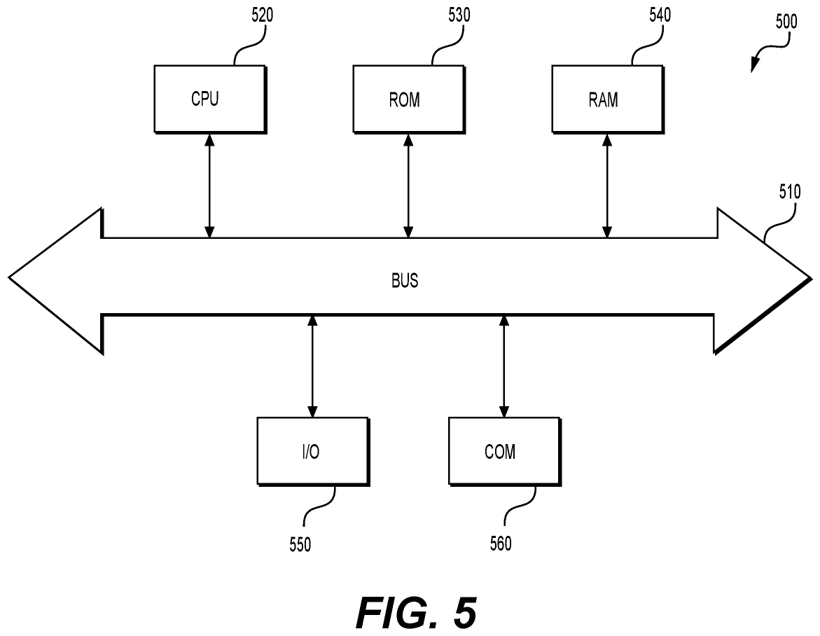

FIG. 5 is a simplified functional block diagram of a computer 500 that may be configured as a device for executing the method of FIG. 2, according to exemplary embodiments of the present disclosure. For example, device 500 may include a central processing unit (CPU) 520. CPU 520 may be any type of processor device including, for example, any type of special purpose or a general-purpose microprocessor device. As will be appreciated by persons skilled in the relevant art, CPU 520 also may be a single processor in a multi-core/multiprocessor system, such system operating alone, or in a cluster of computing devices operating in a cluster or server farm. CPU 520 may be connected to a data communication infrastructure 510, for example, a bus, message queue, network, or multi-core message-passing scheme.

Device 500 also may include a main memory 540, for example, random access memory (RAM), and also may include a secondary memory 530. Secondary memory 530, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 530 may include other similar means for allowing computer programs or other instructions to be loaded into device 500. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 500.

Device 500 also may include a communications interface (“COM”) 560. Communications interface 560 allows software and data to be transferred between device 500 and external devices. Communications interface 560 may include a modem, a network interface (such as an Ethernet card), a communications port, a PCMCIA slot and card, or the like. Software and data transferred via communications interface 560 may be in the form of signals, which may be electronic, electromagnetic, optical, or other signals capable of being received by communications interface 560. These signals may be provided to communications interface 560 via a communications path of device 500, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 500 also may include input and output ports 550 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the general description and the detailed description are exemplary and explanatory only and are not restrictive of the features, as claimed.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for predicting a risk level for the presence of persistent deciduous teeth (PDT) for one or more pets, the computer-implemented method comprising:
receiving, by one or more processors, pet data corresponding to a pet from a user device, the pet data including one or more pet attributes;
based on the one or more pet attributes, determining, by the one or more processors, a result value indicating a PDT attribute weight for each of the one or more pet attributes;
analyzing, by the one or more processors, the result value for each of the one or more pet attributes to determine a PDT risk level, the analyzing including utilizing a PDT risk level prediction algorithm; and
displaying, by the one or more processors, the PDT risk level on one or more user interfaces of the user device.

2. The computer-implemented method of claim 1, the analyzing, by the one or more processors, the result value to determine the PDT risk level further comprising:
receiving, by the one or more processors, one or more pet dental datasets for one or more similar pets from one or more data stores, wherein the one or more similar pets have at least one similar attribute to the pet; and
utilizing, by the one or more processors, the PDT risk level prediction algorithm to determine the PDT risk level by comparing the result value corresponding to each of the one or more pet attributes with the one or more pet dental datasets.

3. The computer-implemented method of claim 1, wherein the pet data includes at least one of: an image of the pet, a medical record corresponding to the pet, pet data input via the user device, or pet data stored in one or more pet data stores.

4. The computer-implemented method of claim 1, wherein the PDT risk level includes a low risk, a medium risk, or a high risk.

5. The computer-implemented method of claim 1, wherein the PDT risk level indicates a probability of the pet being diagnosed with PDT.

6. The computer-implemented method of claim 1, the computer-implemented method further comprising:
determining, by the one or more processors, one or more recommendations based on the PDT risk level; and
displaying, by the one or more processors, the one or more recommendations on the one or more user interfaces of the user device.

7. The computer-implemented method of claim 1, wherein the one or more pet attributes include one or more of: a pet type including a breed, a pet head shape, a pet weight size category, a pet weight condition, a pet body condition, a predicted pet size category, a pet adult weight category, a pet age, a pet medical interaction frequency, a pet plan, or a last medical interaction.

8. The computer-implemented method of claim 7, wherein the pet weight size category includes at least one of: an extra-small category, a small category, a medium-small category, a medium-large category, a large category, or an extra-large category.

9. A computer system for predicting a risk level for the presence of persistent deciduous teeth (PDT) for one or more pets, the computer system comprising:
at least one memory storing instructions; and
at least one processor configured to execute the instructions to perform operations comprising:
receiving pet data corresponding to a pet from a user device, the pet data including one or more pet attributes;
based on the one or more pet attributes, determining a result value indicating a PDT attribute weight for each of the one or more pet attributes;
analyzing the result value for each of the one or more pet attributes to determine a PDT risk level, the analyzing including utilizing a PDT risk level prediction algorithm; and
displaying the PDT risk level on one or more user interfaces of the user device.

10. The computer system of claim 9, the analyzing the result value to determine the PDT risk level further comprising:
receiving one or more pet dental datasets for one or more similar pets from one or more data stores, wherein the one or more similar pets have at least one similar attribute to the pet; and
utilizing the PDT risk level prediction algorithm to determine the PDT risk level by comparing the result value corresponding to each of the one or more pet attributes with the one or more pet dental datasets.

11. The computer system of claim 9, wherein the pet data includes at least one of: an image of the pet, a medical record corresponding to the pet, pet data input via the user device, or pet data stored in one or more pet data stores.

12. The computer system of claim 9, wherein the PDT risk level includes a low risk, a medium risk, or a high risk.

13. The computer system of claim 9, wherein the PDT risk level indicates a probability of the pet being diagnosed with PDT.

14. The computer system of claim 9, the instructions further comprising:
determining one or more recommendations based on the PDT risk level; and
displaying the one or more recommendations on the one or more user interfaces of the user device.

15. The computer system of claim 9, wherein the one or more pet attributes include one or more of: a pet type including a breed, a pet head shape, a pet weight size category, a pet weight condition, a pet body condition, a predicted pet size category, a pet adult weight category, a pet age, a pet medical interaction frequency, a pet plan, or a last medical interaction.

16. The computer system of claim 15, wherein the pet weight size category includes at least one of: an extra-small category, a small category, a medium-small category, a medium-large category, a large category, or an extra-large category.

17. A non-transitory computer-readable medium storing instructions that, when executed by at least one processor, cause the at least one processor to perform operations for predicting a risk level for the presence of persistent deciduous teeth (PDT) for one or more pets, the operations comprising:
receiving pet data corresponding to a pet from a user device, the pet data including one or more pet attributes;
based on the one or more pet attributes, determining a result value indicating a PDT attribute weight for each of the one or more pet attributes;
analyzing the result value for each of the one or more pet attributes to determine a PDT risk level, the analyzing including utilizing a PDT risk level prediction algorithm; and
displaying the PDT risk level on one or more user interfaces of the user device.

18. The non-transitory computer-readable medium of claim 17, wherein the pet data includes at least one of: an image of the pet, a medical record corresponding to the pet, pet data input via the user device, or pet data stored in one or more pet data stores.

19. The non-transitory computer-readable medium of claim 17, wherein the PDT risk level includes a low risk, a medium risk, or a high risk.

20. The non-transitory computer-readable medium of claim 17, the analyzing the result value to determine the PDT risk level further comprising:
receiving one or more pet dental datasets for one or more similar pets from one or more data stores, wherein the one or more similar pets have at least one similar attribute to the pet; and
utilizing the PDT risk level prediction algorithm to determine the PDT risk level by comparing the result value corresponding to each of the one or more pet attributes with the one or more pet dental datasets.

21. A computer-implemented method for predicting a risk level for the presence of persistent deciduous teeth (PDT) for one or more pets, the computer-implemented method comprising:
receiving, by one or more processors, pet data corresponding to a pet from a user device, the pet data including one or more pet attributes;
based on the one or more pet attributes, determining, by the one or more processors, a result value indicating a PDT attribute weight for each of the one or more pet attributes;
analyzing, by the one or more processors, the result value for each of the one or more pet attributes to determine a PDT risk level, the analyzing including utilizing a PDT risk level prediction algorithm;
displaying, by the one or more processors, the PDT risk level on one or more user interfaces of the user device and a corresponding intervention, wherein the intervention includes recommending the removal of one or more teeth, recommending more frequent visits to a medical professional, or recommending an oral health regime in order to decrease the risk of periodontal disease that could be a consequence of PDT; and
performing, by a user, the intervention on the one or more pets.
